(19) United States
(12) Patent Application Publication
(10) Pub. No.: US 2026/0051363 A1
(43) Pub. Date: Feb. 19, 2026

(54) STRUCTURAL RULES FOR DESIGNING MULTI-FUNCTIONAL BIOCATALYSTS

(71) Applicant: Iowa State University Research Foundation, Inc., Ames, IA (US)

(72) Inventors: Ratul Chowdhury, Ames, IA (US); Sakib Ferdous, Ames, IA (US); Priyanshu R. Gupta, Austin, TX (US)

(21) Appl. No.: 19/304,132

(22) Filed: Aug. 19, 2025

Related U.S. Application Data
(60) Provisional application No. 63/684,621, filed on Aug. 19, 2024.

(51) Int. Cl. G16B 15/20; C12N 9/14; C12N 9/96; G16B 35/20

(52) U.S. Cl. CPC G16B 15/20; C12N 9/14; C12N 9/96; G16B 35/20

(57) ABSTRACT

A systematic pipeline is used to extract catalytically active pockets of the most diverse enzyme class—hydrolases, from the PDB database. A process extracts the 38029 hydrolase reactive centers (RC) and collates them into a publicly accessible active site collection (actiome; RC-Hydrolase). The process includes 128M pairwise shape comparisons across RC-Hydrolase using CADSEEK 3D Shape Search Engine to end up with 155,329 instances presented in a available, visually interactive dataset. Allowing comparisons of enzyme reactive centers across functional spaces (EC classification numbers) enables identification of enzyme backbones which can be minimally mutated to accommodate more than one type of catalytic activity to aid rational design of multifunctional enzymes. Such versatile enzyme backbones is leveraged by latest diffusion-based protein design models to design a library of structurally stable multifunctional enzyme pockets. Design of a bifunctional protease-nuclease shown as an example opens up a novel computational recipe for enzyme engineering.

### CROSS REFERENCE TO RELATED APPLICATIONS

This application claims priority under 35 U.S.C. § 119(e) to provisional patent application U.S. Ser. No. 63/684,621 filed Aug. 19, 2024. The provisional patent application is hereby incorporated by reference in its entirety herein, including without limitation: the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant Number OIA2242763 awarded by the National Science Foundation. The government has certain rights in the invention.

### TECHNICAL FIELD

The present disclosure relates generally to enzymes. More particularly, but not exclusively, the present disclosure relates to methods and systems for identifying reactive centers of enzymes and use of the same for combining enzymes for multifunctional uses to catalyze multi-step reaction.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

As the largest and most diverse class of enzymes, hydrolases present an opportunity to explore their conformational diversity, which underpins their varied biological functions. Recent trends necessitate re-evaluating and updating our understanding of the functional and conformational transitions of these enzymes.

Hydrolases are an industrially relevant class of enzymes for their diverse therapeutic and industrial applications. Their basic function is cleaving specific bonds using water (hydrolysis) and can be activated both in vivo as well as ex vivo (such as, small scale well-plates, and large-scale fermenters). There are, in total, 13 different types of hydrolases breaking 13 different types of bonds, making this sub-EC (enzyme classification) class most diverse after oxidoreductases (which has 25 sub-classes, albeit with some ambiguity on the validity of all these sub-classes). Industrially, hydrolases are used in environmental applications such as breaking down plastics and wastewater treatment; detergents and cleaning industry, where proteases and lipases are ingredients in all detergents; food and beverages industry for conversion, extraction, modification, and improvement of flavors; textile industry for finishing, washing, and removal of impurities; breaking down of cellulose and hemicellulose for biofuel production; healthcare industry such as in pharmaceutical manufacturing, drug synthesis, and other biotechnological processes, including DNA manipulation and protein engineering. The global cap of hydrolase is about 6.5 billion USD, and it is expected to grow at a rate of 5.5 percent each year. Growth of innovative biomanufacturing process requires more customized hydrolases.

Enzymes, intricate biological catalysts, have evolved over billions of years to perform specific functions within living organisms necessary for life-sustaining metabolic and signaling processes. This lengthy process of evolution has fine-tuned these enzymes to be highly efficient and specific in their utility. However, when it comes to industrial applications, baseline activity of natural enzymes may not always meet with the specific requirements of various processes. Industrial processes often demand enzymes with enhanced characteristics, such as higher stability, activity under specific conditions, or resistance to certain inhibitors. The focus of hydrolase engineering is to synthesize a biocatalyst that can cleave an intended bond (i.e., be of a desired sub-class) and retain that functionality in an over a wide range of operational condition prompting the need for structurally stable, but customizable backbones for industrial needs. For example, for plastic degradation purpose, PET hydrolases are used. Degradation temperature is typically high to break high-energy bonds in PET. To this end, thermotolerant bacteria that thrive in hot springs bear enzymes that do not denature above 100° C. Computational design of fusion PET hydrolases thermotolerant enzyme structure has been explored as a potential avenue of designing thermotolerant PET. The idea of using enzymes derived from nature as a template for designing industrial enzymes is grounded in the concept of harnessing evolutionary success. Thus far, there have been considerable efforts in designing novel industrial hydrolases. For example—there has been considerable success using directed evolution to design thermostable cellulases, rational design aided by computational docking is used to design substrate promiscuity on serine hydrolases, and metagenomic analyses is to design phthalate degrading hydrolases. However, the competitive biotechnological market necessitates identifying versatile enzyme backbones that houses more than one active site/reaction center for the same or multiple consecutive catalytic reactions. Latest diffusion-based AI-models (e.g., RFDiffusion, Genie2) have shown reasonable performance (with hand-picked experimental proofs) in designing stable fusion proteins which can integrate domains from multiple parent proteins. Regardless, the problem of grafting a specific set of catalytic amino acids/reaction center motifs onto an existing protein scaffold needs sub-Å level accuracy in AI-based structure prediction models (such as, AlphaFold2, RGN2, and ESMFold), otherwise there is a risk of very low catalytic turnover (kcat˜0). Due to the paucity of prior experimental data on reactive center grafting across proteins, it is hard to have a machine learning framework to directly predict the right grafting strategy.

Thus, there exists a need in the art for systems and methods for identifying of enzyme backbones that can be minimally mutated to accommodate more than one type of catalytic activity to aid in the design of multifunctional and/or multistep enzymes with more than one function.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to combine enzymes with identified similar reactive centers to create multifunctional and/or multistep enzymes.

It is still yet a further object, feature, and/or advantage of the present disclosure to identify reactive centers of enzymes that could be minimally mutated to add additional functionality to the enzyme that can now catalyze two-step/three-step (i.e., multistep) reactions.

The systems and/or methods disclosed herein can be used in a wide variety of applications. For example, while certain enzymes, hosts, and functionalities have been included, the same or similar processes could be utilized for not specifically named organisms.

According to some aspects of the present disclosure, a method of identifying two or more enzymes suitable for fusion into a multifunctional enzyme is provided. In some embodiments, the method comprises selecting a first enzyme having a first reactive center; comparing a second enzyme having a second reactive center to the first enzyme; and determining that the first and second enzymes are suitable for fusion if the second reactive center is structurally similar/complementary to the first reactive center.

According to at least some aspects of the disclosure, the comparing step comprises determining the catalytic reactive center similarity (CRCSim) score of the first and second reactive centers. In some embodiments, the first and second reactive centers are deemed structurally similar when the CRCSim score is greater than about 0.8.

According to some aspects of the present disclosure, a method of designing multifunctional enzymes comprises selecting a first enzyme having a first reactive center having a first function; identifying a second enzyme having a second reactive center having a second function that is different than the first function of the first reactive center, wherein the second reactive center includes a similar structure of the first reactive center; and mutating the first enzyme to add the second function to the first reactive center to create a multifunctional/multistep enzyme that performs both the first and second functions.

According to at least some aspects of the disclosure, the similar structure of the first reactive center and the second reactive center comprises similar geometry.

According to at least some aspects of the disclosure, the first reactive center comprises a first catalytic motif and the second reactive center comprises a second catalytic motif. In some embodiments, the multifunctional enzyme has a multifunctional reactive center. In some embodiments, the multifunctional reactive center comprises the first and second catalytic motifs.

According to at least some aspects of the disclosure, the method further comprises designing and/or stabilizing the multifunctional enzyme using a protein hallucinator. In some embodiments, the protein hallucinator comprises ProteinMPNN.

According to at least some aspects of the disclosure, the first and second enzymes are stored in and/or selected from a database.

According to at least some aspects of the disclosure, the amount of similarity between the first and second reactive centers comprises a CRCSim score greater than about 0.8.

According to at least some aspects of the disclosure, the first and/or second enzyme is a hydrolase.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows a workflow to obtain protein active site pockets using precise experimental annotation of catalytic residues.

FIG. 2A shows data statistics of the number of structures across prominent organisms.

FIG. 2B shows data statistics of the number of instances across sub-EC class of interest 3.1-3.6 and others.

FIG. 3A shows the distribution of percent similarity of pairwise active site shapes in the database after 60 percent CRCSim score cutoff in the order of total database, comparison instances where ligand type is different, the source organism super kingdom is different, expression host for the enzyme is different and sub-EC class is different.

FIG. 3B is a heatmap showing EC class matches by similarity.

FIG. 3C shows graphs of log scale compared by RCSim score bins.

FIG. 4 shows two protein structures from different expression hosts—M. smegmatis and S. cerevisiae have structurally similar reactive centers with CRCSim score >0.77 (99^{th} percentile) even though their overall structural similarity is at best modest (85% fold similarity, but 30% global distance match, and >8 Å dissimilarity in atomic coordinates) and sequence similarity being poor (˜60%).

FIG. 5A shows a design pipeline where two protein structures 6JVN of 3.6 (Phosphatase) and 5D6E of 3.4 (Protease) share similarity in their active site shape and the pocket void volume.

FIG. 5B shows the chemical structure of the respective ligands—ligand CJ0 belonging to 6JVN and ligand 94A belonging to 5D6E.

FIG. 5C shows that, although there is little similarity between the two proteins in terms of sequence and structure demonstrated by sequence similarity, RMSD score, TM-score and GDT-TS score, the structure of binding pocket is similar as shown by CRCSim score 0.7 which is 99^{th} percentile.

FIG. 5D provides an active site for sequence 6JVN with the active site residue Tyr7 marked with red, active site for 5D6E with Asp262 and His331 marked with blue, A new pocket is engineered named DES-1 by mutating the Trp119 to His119 using ProteinMPNN by making minimum mutations (indicated by black arrow from top) to conserve the structure of the reactive site to engineer both functionality in a single pocket.

FIG. 5E shows a depiction of the sequence of FIG. 5D.

FIG. 6A is a view of the RC-Hydrolase database showing reactive centers of hydrolases clustered by their shapes.

FIG. 6B is a view of the RC-Hydrolase database showing a PDB accession id search for any reactive center containing with 6JVN as an example.

FIG. 6C is a view of the RC-Hydrolase database showing results retrieved from the query.

FIG. 6D is a view of the RC-Hydrolase database showing a CADSEEK shape search action on the reactive center of 6JVN, which is co-crystallized with the small molecule ligand_CJ0, and internally indexed in RC-Hydrolase with code 200A_6_9606. The suffix. pdb.wrl indicates that the PDB formatted file was converted to a wrl file for CAD operations.

FIG. 6E is a view of the RC-Hydrolase database showing a sample CADSEEK real-time shape search result.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The term “reactive center”, as used herein, refers to the region of an enzyme where substrate molecules bind and undergo a chemical reaction (the active site) as well as the region of about 10 angstrom around the active site that contributes to holding the shape of the reactive center.

The term “residue”, as used herein, refers to a specific monomer within a nucleic acid or protein. Nucleic acid residues comprise adenine (A), guanine (G), cytosine (C), thymine (T), or uracil (U). A protein or polypeptide residue comprises an amino acid.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

To address the lack of unified structural-biochemical rules, the RC-Hydrolase database was created by (a) extracting the reactive centers/active site pockets from 38029 hydrolases from Protein Data Bank cross-validated using experimental annotation of catalytic motifs, and heteroatomic ligands bound to the enzyme, and (b) using CADSEEK for the automated encoding, classification, generation of shape analytics reports, and performing rapid on-the-fly 3D shape search to visually show to a user, all other hydrolases with a CADSEEK 3D shape search engine Reactive Center Similarity (CRCSim)>0.6. RC-Hydrolase serves as the first interactive web-repository and search optimization platform that enables comparison of reactive centers across different enzyme classification numbers (i.e., enzymes with very different functions such as, proteases, and nucleases). A pair of hydrolases with different EC numbers but high CRCSim scores indicate versatile starting backbones where point mutations to the active site can yield multi-functional hydrolase. For example, if an industrial protease—subtilisin, that is optimally secreted from a production host (Bacillus subtilis) in a fermenter, now doubles up as an active nuclease, it would help removing recombinant DNA (adhering to regulatory concerns) from the commercial detergent. Similarly, if the same protease can house more than one protease reactive center, that also aids in catalytic turnover. RC-Hydrolase thus, acts as a repository for experimental use case, and subsequent AI/ML enzyme engineering campaigns.

Clustering active site shapes in context of generative protein design.

Protein data, either sequence or 3D structure format, are analyzed and interpreted by clustering them to groups based on similarity to reduce redundancy, to gain functional insight and efficient searching. Albeit the critical role played by the 3D structure, the most popular and feasible way to assess and analyze a catalytic protein is to align and compare it with other sequences considering there had been larger number of historically prevalent unannotated sequences than experimentally determined structures. BLAST, MMseq2 and Clustal Omega enable the identification and alignment of similar sequences across different organisms, facilitating evolutionary studies and functional annotation. The assumption is that similar sequences take up similar structures and thus perform similar functions. In practice, that is not always the case and there are instances where similar sequences and structure exhibit different functions or different sequences/structures exhibiting same functions. For instance, a small number of mutations will preserve the homologous similarity but can alter the structure of the protein completely resulting in totally different functions. Those mutations in the active sites might drive the functionality to a different direction. Two proteins with similar sequences might behave differently due to their interactions with different partners or because they operate in different cellular contexts. For example—myoglobin and hemoglobin share about 93% sequence similarity, but they transfer oxygen in two different matrixes. Actin, a protein critical for cell structure and motility, although do not have a high degree of sequence similarity, shares a surprising degree of structural similarity with the Hsp70 family of heat shock proteins, which function as molecular chaperones.

G-Protein Coupled Receptors are a large family of proteins with similar structures (seven transmembrane domains) but diverse functions, ranging from sensory perception (like vision and taste) to neurotransmitter and hormone signaling. Let's say there is an enzyme which performs its catalytic function with the help of a catalytic triad. The three amino acids from this triad are far away in sequence but they are held close together by the tertiary structure. Now, if these three amino acids are mutated, the protein will not be able to perform the same function, but still will have high sequence and structural similarity. The position of an amino acid within a protein's three-dimensional structure can affect its role and importance. A cysteine residue might be just a regular structural component in one protein, but in another, it might form a critical disulfide bond that is essential for maintaining the protein's active conformation, for example cysteine in collagens and immunoglobulins. The function of an amino acid residue can be influenced by allosteric sites elsewhere in the protein. A residue that does not seem important for enzyme activity might be crucial for allosteric regulation, affecting the protein's activity in response to binding of a molecule at a distant site.

With the emergence of protein-based large language models (ESM2, AminoBERT), and family sequence parsing through transformer networks (Evoformer of AlphaFold2, ESMFold), AI-based prediction of enzyme structures has opened doors towards incorporating 3D-structural information along with sequence to cluster proteins with higher functional fidelity. Available data points used to train these models are the existing protein structure and sequences shaped by evolution. So, sequence-based structure or function prediction is not free from bias. Given the changed landscape of the paradigm, researchers developed clustering algorithms which can group unannotated 3D structures based on functions, for example-FoldSeek. But, clustering structures have their own caveats. Chains of amino acids arrange themselves in 3D space leading to emergence of catalytically active precise binding pocket/groove geometries (active sites/reactive centers) which accommodate substrates and cofactors to perform designated catalytic breakdown, often spanning more than one chain. Essentially, the function of the enzyme depends on a very focused chemical sub-surface in the enzymatic fold. The rest of the enzyme's bulk evolves to hold the catalytic sub-surface in the right orientation and even absorb evolutionary mutations without undergoing structural change to the catalytic pocket. In other words, the action of the enzyme depends on how the catalytically relevant residues are oriented in 3D. The diversity of the total protein structure universe poses a significant challenge, as much of the structure is not directly informative about the core reactions being catalyzed. Consequently, clustering these diverse structures suggests numerous possibilities, complicating the selection of ideal candidates for engineering. It would not be an exaggeration to say the real driver between protein function is neither the sequence nor structure, rather a very small group of amino acids held at a certain orientation in a favorably shaped surface. As most of the functions happen in a relatively small concave region, it makes more intuitive sense to cluster the active site only. For special cases of natural enzymes such as extremophiles, the sequence is not related evolutionary to the known animal kingdom, rather originated in a specific extreme environment. As these enzymes cannot be characterized by homologous analysis, a very different scheme is needed to analyze the functions of such enzymes, which will consider the core component responsible for the catalytic action. A computational analysis of the surface of that region can be helpful in analyzing, comparing, and designing protein structures.

Algorithms for comparing active site pockets includes traditional methodologies like clique detection, triplet and quadruplet matching, pairwise and template-based alignment, as well as machine learning methods CNN, autoencoders, and Random Forest. Although there are several databases, most of these are currently out of maintenance and need additional coding to execute them and most importantly none of them come with a publicly accessible website. A visual, interactive, publicly available database for matching active sites with high structural similarity across different enzyme classes (functions) for a user preferred host organism is absent. Some of the reasons can be attributed to the cost of maintaining the database and the challenge of updating them frequently. On the contrast, the sequence-based databases are rich, robust, regularly maintained and updated. 3D structure databases of proteins (AlphaFoldDB, PDB) offer homology-based overall structural similarity, thus complementing them with a substructure searching and matching enables functional clustering of reactive centers of enzymes, and other key pockets across proteins (RC-Hydrolase). This task requires carefully scanning pockets of importance which often span one or more chains within/between multiple interacting proteins.

Rational design efforts designed active sites with structural modeling tools accounting in isolation or some combination thereof of amino acid sequence, homology, and solvent accessible surface. These tools were limited by the number of maximum mutations can be explored as the searching the sequence space becomes combinatorically explosive, and NP-complete. While sporadic successes have been obtained using integer (IPRO+/−) and Monte Carlo sampling (Rosetta3), recent advent of generative AI to design proteins (RFDiffusion, Genie2) have made this a reasonably tractable problem (with caveats such as—assuming AlphaFold2-predicted structure is equivalent to the ground-truth experimental structures). Using generative AI, it is theoretically possible to hallucinate the neighboring residues around the active site, to design a desired enzyme. Such efforts can glean reasonable target templates from RC-Hydrolase to drive the designs towards pocket geometries that are known to be functional for the intended action. It is envisioned that an effective way to harness the AI-power in protein engineering is carefully leveraging several tools with a common goal, rather than designing a one-stop-solution for all enzyme engineering goals given how drastically different two enzymes can be in (a) fold-stability relationships, (b) set of interacting molecules, and (c) biochemical micro/macroenvironments at which they are expected to function. Functionally, bio-aware data collection and classification is a necessary prior to all downstream activities. Much effort is warranted in clustering the shapes of active sites/reaction centers in place of the total structure or sequence. To date, very few efforts have been observed on clustering reaction centers. Protein surface shape is also discussed in literature for the purpose of comparison, classification and identification without any downstream design strategy or publicly accessible visual repository.

### The Utility of RC-Hydrolase Database and CADSEEK 3D Shape Search and Analytics Engine

The most important attribute of an industrial enzyme is the catalytic efficiency—or the kcat/kM, is the number of molecules of substrate converted to product per active site per unit time (kcat) when given the enzyme's affinity towards the substrate (KM). Thus, the ability to engineer same/different reaction center (active site) of interest in seemingly unanalytic regions of the enzyme's pocket can enhance the turnover number and utility of an enzyme. The RC-Hydrolase database will also aid in designing multifunctional enzymes by providing starting points for de novo design of fusion enzymes which contain intended catalytic reaction centers thus pushing the limits of AI-guided functional enzyme design. The production cost of a commercial enzyme (protein product) is driven by the expression efficiency in host as well as the catalytic properties of the enzyme. A far-reaching biotechnological and experimental utility of RC-Hydrolase is to identify enzymes from other expression hosts with a desired activity, and re-structure the target reactive center with minimal edits (mutations) in the enzyme from the standard expression host. This is likely to augment the chances of producing the engineered enzyme without compromising expression levels. Also, sub-surface shape matching algorithm can direct us to down-select alternative candidate expression host with comparable yields or to identify/design minimal (shorter) enzymes which can bring down the cost and uncertainties of producing a relatively larger protein. Finally, CRCSim scores can aid in switching functionalities of different enzymes within the same host, thereby offering a new route to alter reaction fluxes—i.e., enzyme structure-guided metabolic engineering.

Therefore, as will be understood, the present disclosure includes an outline for precise experimental annotations of catalytic residues aggregated in interpro and coupling that with structural info from pdb to pinpoint the active site. Besides active sites, the disclosure includes a visual, interactive, publicly available database for matching active sites with high structural similarity across different enzyme class num (functions) for a user preferred host organism. This is important for identifying versatile starting points for engineering enzymes with more than one active site that controls the same or different functions; a strategy to extract catalytically relevant sub surface from a protein structure. This method extracted sub-surface or catalytic pockets of 16,036 hydrolase structures of 6 EC class of interest—3.1-3.6 (namely—esterase, glycosylase, etherase, peptidases, C-N bonds other than peptide bonds, and Acid anhdrides) across 4 domains of life, namely Archaea, Eukaryotes, Fungai, and Bacteria. The shapes were clustered using a custom algorithm (discussed herein). Onwards, the disclosure includes the shape mapping algorithm and creation of a hydrolase database and outline how this can be leveraged along with generative AI based protein design tools, such as ProteinMPNN, using one test example.

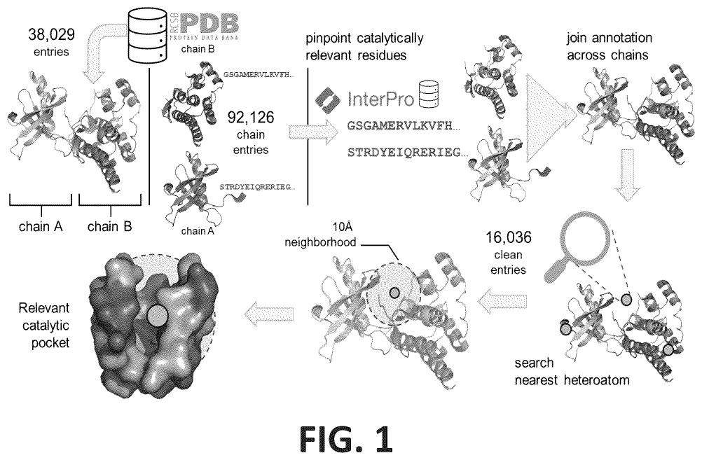

FIG. 1 shows a workflow to get the protein active site pockets using precise experimental annotations of catalytic residues. 38029 PDB structures were obtained from RCSB databank, the sequences corresponding to each of the chains were extracted, and these sequences were sent to the Interpro database to identify the conserved domain and functions. The conserved residues were traced back to the structure, the ligand which contains the greatest number of conserved residues within a defined radius is taken as a centroid and centering it all the residues within 10 Å distance is taken which defines the pocket.

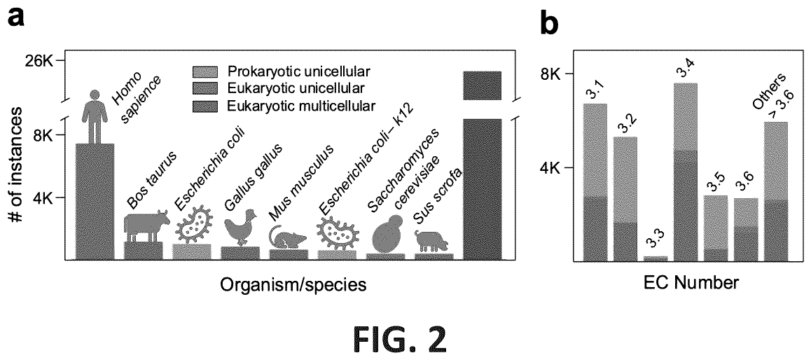

In total, 8029 hydrolases have been reported to date (September 2023) in humans, with their sizes ranging from 6 aa to 3905 aa. Hydrolase 3.4 happens to be the most crystallized member of human hydrolases (41%), which probably alludes to the research focus on proteases for digestive, immune-therapeutic, tissue engineering efforts that prompted people to crystallize human proteases 45. On the contrary—in the bacterial world, 3.4 is the most popular crystal member—which corresponds to the research thrust on antimicrobial peptides. Instances are left which are arranged according to sub-EC class constituting our primary list. Most of them belong to 3.1-3.6 sub-EC class comprising a total number of 23415. These structures are from different organisms, the highest one from Homo Sapiens. Other prominent members are shown in FIGS. 2A and 2B.

### Similar Chemically Relevant Surfaces Across Organism and EC Class

It was initially hypothesized that the function and performance of an enzyme is driven by the shape of the catalytically relevant sub surface. Thus, enzymes belonging to the same sub-EC class acting on similar substrate will have similar active surface shape and different enzymes will have different shapes. If there is a cross sub-EC class match, this is essentially the scope of designing multifunctional enzymes. Analyzing the active site shape provides insight on both these categories. All against all matching of 16036 instances consists of 128,568,630 comparisons, with the cut off at 60 percent CRCSim score between shapes found out by CADSEEK we get 155329 matches which is 0.12% of the dataset. This indicates diversity of the substrate type and resulting low similarity of the pocket shape overall.

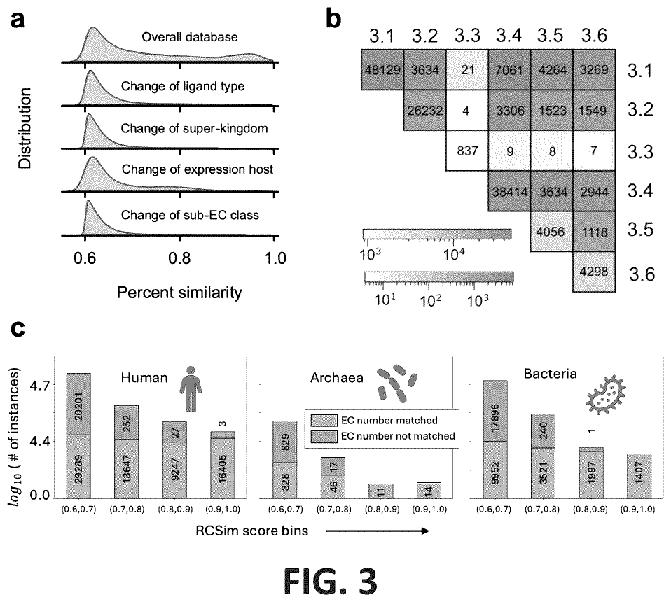

Cases where the EC class matches among the similarity dominate as shown by a separate blue log scale along the diagonal of the heatmap (see, e.g., FIG. 3B). Most of the comparison lies close to 60% as it was the cutoff. All the ligands and host organisms were divided into some broad classes. The instances where there is a class mismatch, the number of matches reduces even further. But these are the valuable cases where cross design switching host or ligand is possible. For designing cross functional or multifunctional enzymes cases where there is a shape similarity across EC class could be a starting point. The esterase class 3.1 is most diverse in that respect, having more similarity with the other 5 EC classes discussed. A possible reason might be the broad definition of the EC class 3.1 consisting of nucleases, phosphatases and lipases thus resulting similarity in shape with other EC classes as their substrates shapes overlap.

As shown in FIGS. 3A-3D, distribution of percent similarity of pairwise active site shapes in the database after 60 percent CRCSim score cutoff in the order of total database, comparison instances where ligand type is different, the source organism super kingdom is different, expression host for the enzyme is different and sub-EC class is different. The higher the similarity of the pocket across domain is the better candidate for applying further in silico design and expression (FIG. 3A). The number of instances which share more than 60 percent CRCSim score within or across EC class, the diagonal elements which are comparison between same EC class are marked with blue log scale color bar, the off-diagonal comparison which share inter EC-class comparison are expressed with red log scale color bars (FIG. 3B).

### Identifying Similar Pockets Across Expression Hosts

Protein expression hosts are crucial due to their impact on production yield, efficiency, and protein functionality. High-yield hosts like E. coli are cost-effective and rapid but may lack necessary post-translational modifications such as glycosylation, which are essential for the functionality of certain proteins and are better performed by mammalian cells like CHO cells. The choice of host affects the biological activity of the protein, with some hosts better facilitating proper folding and disulfide bond formation. Cost and scalability are also considerations, with bacterial systems generally cheaper and faster than mammalian systems. Additionally, compatibility with the protein of interest is important, as some proteins may be insoluble or toxic in certain hosts. Regulatory approval often favors well-established hosts like CHO cells for therapeutic proteins. The ease of protein purification can also vary, with hosts like yeast and insect cells (e.g., Pichia pastoris, Sf9 cells using Baculovirus) providing a balance between cost and the ability to perform complex modifications. Selecting the right host ensures sufficient production of the protein with the desired structure and functionality, impacting the success of research and industrial applications.

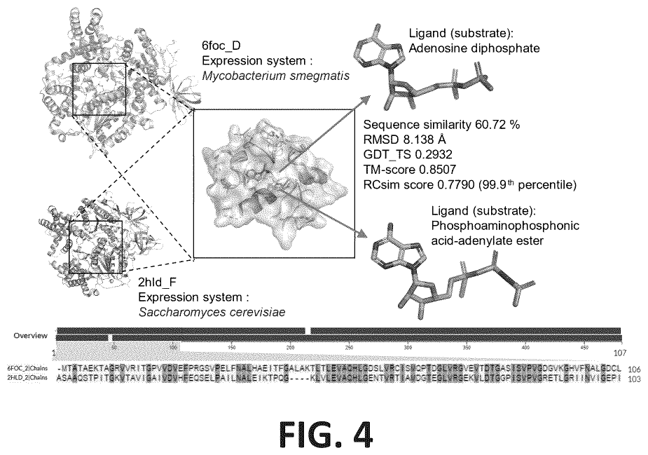

Having a database that identifies similar binding pockets can be useful for pinpointing similar catalytic pocket shapes across various expression hosts for selecting the most suitable one for producing a protein with the desired properties, ensuring proper folding and post-translational modifications. For instance, if a protein requires specific glycosylation patterns, the database can identify mammalian hosts like CHO cells that perform these modifications correctly. This approach also allows for the selection of hosts across domains of life, for example-Prokaryote, Eukaryote and Fungi that maximize yield and minimize production costs. Using our database we have identified a pair of such pockets which function as ATPase from two different expression hosts M. smegmatis (Prokaryotic system) and S. cerevisiae (Eukaryotic system) are 6FOC and 2HLD which share low overall similarity in structure and homology but high CRCSim score (FIG. 4).

FIG. 4 shows two protein structures from different expression hosts—M. smegmatis and S. cerevisiae, which have structurally similar reactive centers with a CRCSim score >0.77 (99^{th} percentile) even though their overall structural similarity is at best modest (85% fold similarity, but 30% global distance match, and >8 Å dissimilarity in atomic coordinates) and sequence similarity being poor (˜60%). Such pairs would be categorized as dissimilar by every metric besides CRCSim score even though they are capable of catalyzing substrates of similar shapes and functional chemistry.

### Designing Multifunctional Enzymes Using RC-Hydrolase Database and ProteinMPNN

From the database, a pair of enzymes are taken which have different sub-EC class, 6JVN belongs to enzyme class 3.6, phosphatases and 5D6E belonging to enzyme class 3.4 Proteases. Although they have very low sequence similarity (19.6%), GDTTS score (0.0795), and a high RMSD score (6.9 Å), their active site shapes match with a high CRCSim score (0.7164 which is at the 99th percentile for matching pairs). Further, the catalytic residue in 6JVN is the Tyr7 and that in 5D6E is the Asp262 and His331. However, a high CRCSim means these two pockets are ideal design candidates for accommodating both binding sites. Although the Asp262 and His331 is distant in sequence of 5D6E, it is hypothesized that a close Trp119 in the 6JVN sequence can be altered to His119 to form the catalytic pair residing in the same orientation and distance, thus can perform the same catalytic action.

Following this mutation, the process leveraged ProteinMPNN to design new sequences that can adopt this amino acid transformation of Trp119His in the crystal structure of PDB 6JVN. ProteinMPNN was used to mutate only the residues involved in the binding pocket while keeping the three catalytic residues (Try7 for enzyme class 3.6+Asp262 and His119 for enzyme class 3.4 functionality) as well as the remaining protein not part of the pocket to be fixed. Using a sampling temperature of 0.3, 15 novel sequences were generated. Only one of the 15 sequences had a global score >1.

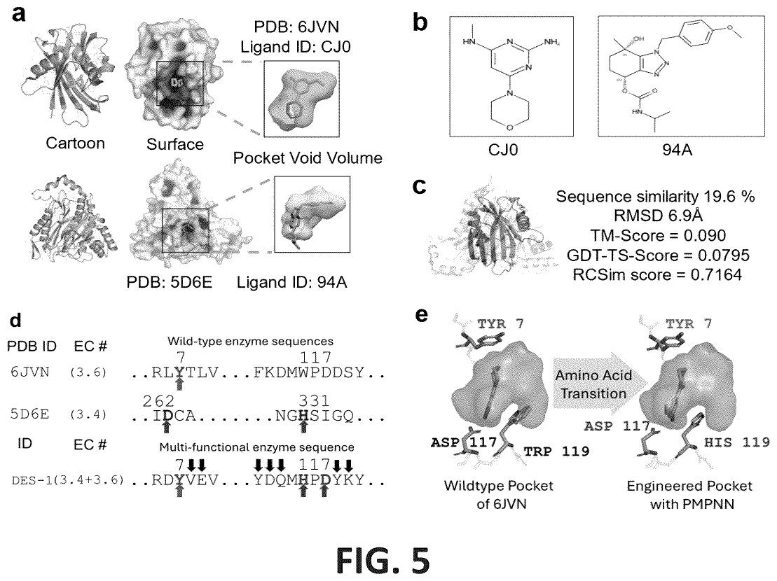

FIGS. 5A-5E show a design pipeline proposition for novel engineered enzyme using RC-Hydrolase database and ProteinMPNN, wherein FIG. 5A shows two protein structures 6JVN of 3.6 (Phosphatase) and 5D6E of 3.4 (Protease) share similarity in their active site shape and the pocket void volume. FIG. 5b shows a chemical structure of the respective ligands-ligand CJ0 belonging to 6JVN and ligand 94A belonging to 5D6E. FIG. 5C shows that, although there is little similarity between the two proteins in terms of sequence and structure demonstrated by sequence similarity, RMSD score, TM-score and GDT-TS score, the structure of binding pocket is similar as shown by CRCSim score 0.7 which is 99^{th} percentile. FIG. 5D shows the active site for sequence 6JVN with the active site residue Tyr7 marked with red, active site for 5D6E with Asp262 and His331 marked with blue. A new pocket is engineered named DES-1 by mutating the Trp119 to His119 using ProteinMPNN by making minimum mutations (indicated by black arrow from top) to conserve the structure of the reactive site to engineer both functionality in a single pocket.

### RC-Hydrolase Database for Hydrolase Reactive Centers

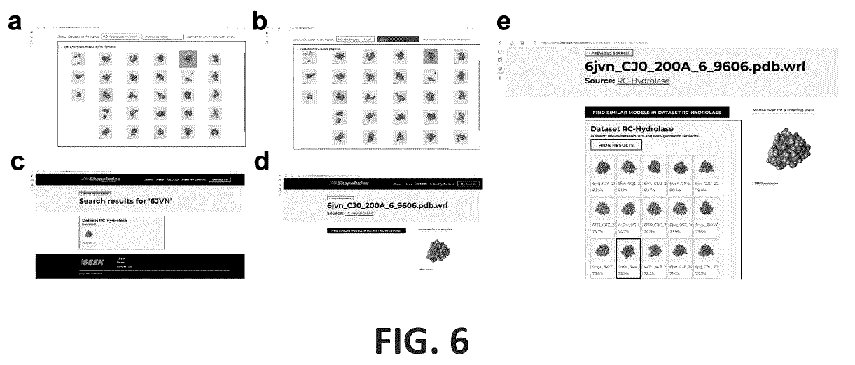

RC-Hydrolase is the first publicly available database (available through https://www.3dshapeindex.com) of its kind to have the catalytic binding pockets of Hydrolase enzymes of sub-EC class 3.1-3.6 (see, e.g., FIGS. 6A-6E). Here, reactive centers of hydrolases are clustered as per their explicit structural similarity with an implicit knowledge of the chemical groups that line these pockets. CADSEEK instantaneous pairwise shape match engine enables rapid search and retrieval of identical or similar pocket geometries. The color depth of each pocket in the interactive dataset indicates the number of candidate enzymes within the CADSEEK shape classification hierarchy; deeper the color the more versatile that pocket is for designing to convert or accommodate reaction by other pockets.

FIGS. 6A-6E show the process mining for similarly shaped pockets in RC-Hydrolase database where FIG. 6A shows reactive centers of hydrolases clustered by their shapes, FIG. 6B shows a PDB accession id search for any reactive center containing with 6JVN as an example, FIG. 6C shows results retrieved from the query, and FIG. 6D shows a CADSEEK shape search action on the reactive center of 6JVN, which is co-crystallized with the small molecule ligand_CJ0, and internally indexed in RC-Hydrolase with code 200A_6_9606. The suffix. pdb.wrl indicates that the PDB formatted file was converted to a wrl file for CAD operations, and FIG. 6E shows a sample CADSEEK real-time shape search result.

Hydrolases play a crucial role in industrial applications, making up nearly 75% of all enzymes used in industry. Among these, carbohydrates, proteases, and lipases are the most prevalent, together representing more than 70% of the enzyme market. Their widespread use underscores their importance in various industrial processes. Numerous techniques have emerged for experimentally or computationally determining whole protein structures from sequences. However, reaction centers of biocatalytic proteins (enzymes) are represented by a very small set of not necessarily contiguous residues folded into spatial proximity. The present disclosure constructs and leverages a large structural database of structural folds of such reaction centers from all experimentally reported hydrolase structures till date, to effectively design and engineer novel multi-functional enzymes. Utilizing CADSEEK, RC-Hydrolase allows users to down-select candidate easy-to-engineer hydrolase starting backbones, say, a protease, which share a high degree of similarity (CRCSim>0.8) in their reactive center (active site) geometry with a nuclease. By providing information on a starting hydrolase type, specific substrate, and expression host organism, one can identify other similar looking but differently functional hydrolase reactive centers. This thereafter enables identification of hydrolases, which, when minimally mutated can host more than one catalytic motif within the same active site resulting in a multi-functional fusion hydrolase. Careful selection and design of such hydrolase backbones from reliable industrial expression host organisms can lead to design of highly expressing multi-functional hydrolases. Such designed multi-functional reaction centers can be further stabilized using protein hallucinators (ProteinMPNN). The utility of this also extends onto taking stable non-enzymatic backbone and endowing them with catalytic motifs (such as design of catalytic antibodies) and also expanding our RC-Hydrolase database to include reaction centers of AlphaFold2-predicted hydrolases (4.33M entries as per UniProt).

In the biotechnological industry, single enzymes with multiple functions have garnered significant interest due to their versatility and efficiency. These multifunctional enzymes are designed by grafting different hydrolase catalytic sites onto one enzyme molecule, enabling them to catalyze various reactions simultaneously largely through heuristics or directed evolution. This reduces the need for multiple enzymes and simplifies the purification process, cutting down on costs and processing time. For instance, a single enzyme could be engineered to possess both protease and lipase activities, allowing it to break down both proteins and fats in a single step. This capability is particularly valuable in industries such as food processing, detergent manufacturing, removal of recombinant DNA from protein products, removal of impurities from drugs, and biofuel production, where complex substrates are common. Additionally, these enzymes can be tailored to operate under specific conditions, enhancing their stability and efficiency in industrial applications. This innovation represents a significant advancement in enzyme technology, offering a more sustainable and cost-effective solution for various industrial processes. The present disclosure pieces out the most functional (reaction) center of enzymes and uses that as a metric to prepare a publicly available, interactive website to compare with other enzymes with set thresholds of similarity.

For example, starting with C12 (or shorter) acyl ACPs, sequential desaturation (of specific C—C bonds) followed by thioesterization (to replace ACP with an acid group), and final P450 hydroxylation (at target C-positions)—leads to formation of x-hydroxy-alkenoic acids (i.e., Trifunctional Fatty Acids—TFAs). TFAs with correct chain length, double-bond location, and hydroxylation are useful as monomers for additive manufacturing of biodegradable polymers. TFAs find length-specific downstream uses. Short chain TFAs (C8 or lower) exhibit high crystallinity and low solubility in organic solvents, suitable for biodegradable packaging in agriculture and cosmetics, while the longer chain TFAs have elastomeric behavior suitable for medical sutures and drug delivery systems. TFAs serve as precursors and additives for lubricants, food grade formulations, resins, emulsifiers and candles. A single multi-domain, three-step enzyme will enable tunability of intermediate specificity and high conversion of target TFAs. Three-individual enzyme blends (i.e., added one after another) have already demonstrated lack of control on product enrichment, and leads to leaky production of target molecule as the intermediates swim away (diffuse) from the next enzyme which is added after a time gap. Plus, there are a lot of unwanted side reactions to this current implementation. Also, three different enzymes when added one after another can lead to mutual degradation, and deactivation of one enzyme by another (as is the case for known industrial hydrolases). Our proposed design of a single fusion enzyme that packs in more than one function will remedy all these gaps. This would unlock frontiers in synthetic biology through fundamental sequence-structure-activity underpinnings in enzymes and metabolic strain design by enabling sequential conversions in a single enzyme. A unified structural theory of enzyme activity gleaned will reduce our reliance on heuristics and high-throughput experiments enabling more rational, predictive design of bio-manufactured enzymes.

### Pipeline for Extracting Catalytically Relevant Sub-Surface

Methods and systems for extracting catalytically relevant sub-surfaces are also part of the present disclosure. A goal of the present disclosure is to identify the conserved domain or substrate binding site in the protein structure. Conserved domain in sequences is annotated in protein sequences, which can be accessible through several databases, but such annotations are not available for protein structure. Each PDB file contains several chains of protein along with heteroatoms. Conserved domain and hetero atom location is taken as a guide to mark regions of the protein surface as catalytic and identify as pocket. The structure file or PDB file is used to start, and the following steps may be part of the process.
Step 1. For each chain, the sequence is extracted and conserved domains in the chains are annotated with the help of ‘Interpro database’. If an annotation is found those residues are noted.
Step 2. Proteins are structurally and functionally very diverse. That is why the definition of the pocket must be crafted carefully to avoid most corner cases. The present disclosure would like to identify the region which is closest to a ‘center of activity’. Other than conserved residues, hetero atoms can be an indicator for locating potential pockets. Heteroatoms other than water are mostly ions of metals which serve as co factor or substrate on which the protein acts or binds. Possible potential pitfalls are—conserved regions in the structure can span over multiple chains, for example in the case of dimers. That is why centroid of the conserved region cannot be a choice for pocket. We also cannot choose pocket on one form each chain basis because multiple chains can contribute to the combined action of the protein where none of them are homologue/dimer. Rather, for each hetero atom present in the PDB file, a search radius of 20 angstrom is taken and the number of conserved residues in that range is counted. The hetero atom with the greatest number of conserved residues in the search radius is considered as the centroid of the pocket.
Step 3. After the principal heteroatom is marked, all the residues around 10 angstrom is extracted and defined as the pocket. The radius is selected by an educated guess taking into consideration that no major contributor to the shape is missed and the pocket is not so big as it gets difficult to match. A fraction of the total number of residues in the chain or the total structure also is not a great choice, as there are protein instances where the total chain acts as a domain and where the domain is a very small part of a chain.

### HydrolaseDB: Database Creation

To create the HydrolaseDB database, we start with the PDB database. As of 1 Jul. 2024, there used to be 40171 instances of known hydrolase structures on three important spans of life—archaea, eukaryote, and bacteria, with 26143 single chained, 14028 more than one chain, with size range of hydrolases from 6 to 4426. Notable catalytic mechanisms by which they act is—acting as a nucleophile with catalytic triad formation (serine protease, cysteine protease etc.), activating water molecule with coordinated metal ions (Matrix Metalloproteinases (MMPs)). However, a curated Hydrolase active site pocket database was not available for readily, so one was constructed using the method provided herein. The high sequence and structural similarity will ensure ease of expression using microbial hosts owing to their similar amino acid compositions, and backbone integrity. Among these there are instances of structure with broken chains, unknown amino acid sub sequence, corrupted files which do not serve intended purpose. After excluding those, pocket structure is made according to the method outlined herein for the sequences where Interpro50 annotation of conserved domain is found. Interpro is a conglomeration of several such databases. Interpro has a built-in engine for Hidden Markov Model based prediction of protein functional and conserved regions.

CADSEEK 3D Shape Search and Analytics Engine for Automated Encoding and Classification of RC-Hydrolase

CADSEEK is a highly efficient and scalable 3D shape search engine capable of processing any type of 3D digitized shape. It extracts a shape code from the digitized geometry and automatically builds a classification without AI training. This enables users to search for identical or similar 3D shapes in under 2 milliseconds, optimizing the utilization and mining of 3D digital assets.

CADSEEK's algorithms mathematically codify a 3D shape by converting it into a boundary value problem. The solution function of the boundary value problem is expressed in series form, where the coefficients of the series expansion are unique representatives of the boundary conditions. The 3D shape surfaces are used as the boundary conditions for the series expansion solution where an electrostatic charge distribution over the 3D object surfaces simulates the boundary conditions. The field satisfies Poisson's equation for which the solution is unique and non-invertible for a given boundary condition51. Extracted shape codes are then automatically classified by CADSEEK's algorithms to generate a search index.

RC-Hydrolase dataset consisting of >16 k 3D protein pocket shapes (https://www.rc-hydrolase.onrender.com). Analysis time on a on a single Quad core i7 or similar) was as follows: extracting RC-Hydrolase 3D shape codes took 4 hours, the automated classification to generate a searchable index required 10 minutes and the 3D shape analytics report for the full dataset was generated in under 4 seconds. CADSEEK shape coding can be threaded and can be parallelized across multiple workstations or computers.

### Software, Computer System, and Network Environment

Some embodiments described herein make use of computer algorithms in the form of software instructions executed by a computer processor. In some embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), convolutional neural network (CNN), random forest, decision trees, support vector machines, and the like, in order to determine, for a given input, one or more output values. In some embodiments, the input comprises alphanumeric data which can include numbers, words, phrases, or lengthier strings, for example. In some embodiments, the one or more output values comprise values representing numeric values, words, phrases, or other alphanumeric strings. In some embodiments, the one or more output values comprise an identification of one or more response strings (e.g., selected from a database).

For example, a machine learning module may receive as input a textual string (e.g., entered by a human user, for example) and generate various outputs. For example, the machine learning module may automatically analyze the input alphanumeric string(s) to determine output values classifying a content of the text (e.g., an intent).

In some embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In some embodiments, once a machine learning module is trained, e.g., to accomplish a specific task such as identifying certain response strings, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In some embodiments, machine learning modules may receive feedback, e.g., based on user review of accuracy, and such feedback may be used as additional training data, to dynamically update the machine learning module. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC)).

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, some modules described herein can be separated, combined or incorporated into single or combined modules. Any modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

While the methods and systems of present disclosure has been particularly shown and described with reference to specific preferred embodiments, it should be understood that various changes in form and detail may be made therein without departing from the spirit and scope of the present disclosure.

### AI/Machine Learning

Many statistical classification techniques are suitable as approaches to perform the classification described herein. Such methods include but are not limited to supervised learning approaches.

Commonly used supervised classifiers include without limitation the neural network (e.g., artificial neural network, multi-layer perceptron), support vector machines, k-nearest neighbors, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with methods according to the disclosure include quadratic classifiers, k-nearest neighbor, boosting, decision trees, random forests, neural networks, pattern recognition, Bayesian networks and Hidden Markov models. Other classifiers, including improvements or combinations of any of these, commonly used for supervised learning, can also be suitable for use with the methods described herein.

Classification using supervised methods can generally be performed by the following methodology:
1. Gather a training set. The training samples are used to “train” the classifier.
2. Determine the input “feature” representation of the learned function. The accuracy of the learned function depends on how the input object is represented. Typically, the input object is transformed into a feature vector, which contains a number of features that are descriptive of the object. The features may include clinical features of a patient or subject.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g., classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.

In some cases, the individual features are clinical features. In some cases, the clinical feature is a normalized value, an average value, a median value, a mean value, an adjusted average, or other adjusted level or value.

Once the classifier (e.g., classification model) is determined as described above (“trained”), it can be used to classify a sample, e.g., clinical features that are analyzed or processed according to methods described herein.

The trained model and the associated machine learning and application of the model will utilize processors, modules, memories, databases, networks, and potentially user interfaces to show the results and allow changes to be made.

In communications and computing, a computer readable medium is a medium capable of storing data in a format readable by a mechanical device. The term “non-transitory” is used herein to refer to computer readable media (“CRM”) that store data for short periods or in the presence of power such as a memory device.

One or more embodiments described herein can be implemented using programmatic modules, engines, or components. A programmatic module, engine, or component can include a program, a sub-routine, a portion of a program, or a software component or a hardware component capable of performing one or more stated tasks or functions. A module or component can exist on a hardware component independently of other modules or components. Alternatively, a module or component can be a shared element or process of other modules, programs, or machines.

The system will preferably include an intelligent control (i.e., a controller) and components for establishing communications. Examples of such a controller may be processing units alone or other subcomponents of computing devices. The controller can also include other components and can be implemented partially or entirely on a semiconductor (e.g., a field-programmable gate array (“FPGA”)) chip, such as a chip developed through a register transfer level (“RTL”) design process.

A processing unit, also called a processor, is an electronic circuit which performs operations on some external data source, usually memory or some other data stream. Non-limiting examples of processors include a microprocessor, a microcontroller, an arithmetic logic unit (“ALU”), and most notably, a central processing unit (“CPU”). A CPU, also called a central processor or main processor, is the electronic circuitry within a computer that carries out the instructions of a computer program by performing the basic arithmetic, logic, controlling, and input/output (“I/O”) operations specified by the instructions. Processing units are common in tablets, telephones, handheld devices, laptops, user displays, smart devices (TV, speaker, watch, etc.), and other computing devices.

The memory includes, in some embodiments, a program storage area and/or data storage area. The memory can comprise read-only memory (“ROM”, an example of non-volatile memory, meaning it does not lose data when it is not connected to a power source) or random-access memory (“RAM”, an example of volatile memory, meaning it will lose its data when not connected to a power source). Examples of volatile memory include static RAM (“SRAM”), dynamic RAM (“DRAM”), synchronous DRAM (“SDRAM”), etc. Examples of non-volatile memory include electrically erasable programmable read only memory (“EEPROM”), flash memory, hard disks, SD cards, etc. In some embodiments, the processing unit, such as a processor, a microprocessor, or a microcontroller, is connected to the memory and executes software instructions that are capable of being stored in a RAM of the memory (e.g., during execution), a ROM of the memory (e.g., on a generally permanent basis), or another non-transitory computer readable medium such as another memory or a disc.

Generally, the non-transitory computer readable medium operates under control of an operating system stored in the memory. The non-transitory computer readable medium implements a compiler which allows a software application written in a programming language such as COBOL, C++, FORTRAN, or any other known programming language to be translated into code readable by the central processing unit. After completion, the central processing unit accesses and manipulates data stored in the memory of the non-transitory computer readable medium using the relationships and logic dictated by the software application and generated using the compiler.

In one embodiment, the software application and the compiler are tangibly embodied in the computer-readable medium. When the instructions are read and executed by the non-transitory computer readable medium, the non-transitory computer readable medium performs the steps necessary to implement and/or use the present invention. A software application, operating instructions, and/or firmware (semi-permanent software programmed into read-only memory) may also be tangibly embodied in the memory and/or data communication devices, thereby making the software application a product or article of manufacture according to the present invention.

The database is a structured set of data typically held in a computer. The database, as well as data and information contained therein, need not reside in a single physical or electronic location. For example, the database may reside, at least in part, on a local storage device, in an external hard drive, on a database server connected to a network, on a cloud-based storage system, in a distributed ledger (such as those commonly used with blockchain technology), or the like.

It is envisioned that the machine learned model and any of the training of the same could include cloud computing. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service.

A cloud computing environment is service oriented with a focus on statelessness, low coupling, modularity, and semantic interoperability. At the heart of cloud computing is an infrastructure comprising a network of interconnected nodes.

As noted, the training model could be implemented on a user interface. The interface could also be a point on introduction of data, such as training data or test data to compare to the trained model for analysis. The results of the comparison could then be shown on a user interface.

A user interface is how the user interacts with a machine. The user interface can be a digital interface, a command-line interface, a graphical user interface (“GUI”), oral interface, virtual reality interface, or any other way a user can interact with a machine (user-machine interface). For example, the user interface (“UI”) can include a combination of digital and analog input and/or output devices or any other type of UI input/output device required to achieve a desired level of control and monitoring for a device. Examples of input and/or output devices include computer mice, keyboards, touchscreens, knobs, dials, switches, buttons, speakers, microphones, LIDAR, RADAR, etc. Input(s) received from the UI can then be sent to a microcontroller to control operational aspects of a device.

The user interface module can include a display, which can act as an input and/or output device. More particularly, the display can be a liquid crystal display (“LCD”), a light-emitting diode (“LED”) display, an organic LED (“OLED”) display, an electroluminescent display (“ELD”), a surface-conduction electron emitter display (“SED”), a field-emission display (“FED”), a thin-film transistor (“TFT”) LCD, a bistable cholesteric reflective display (i.e., e-paper), etc. The user interface also can be configured with a microcontroller to display conditions or data associated with the main device in real-time or substantially real-time.

Any components of the system could be connected via network or other communication protocol to transfer information, communicate with other systems, or provide other connectivity. In some embodiments, the network is, by way of example only, a wide area network (“WAN”) such as a TCP/IP based network or a cellular network, a local area network (“LAN”), a neighborhood area network (“NAN”), a home area network (“HAN”), or a personal area network (“PAN”) employing any of a variety of communication protocols, such as Wi-Fi, Bluetooth, ZigBee, near field communication (“NFC”), etc., although other types of networks are possible and are contemplated herein. The network typically allows communication between the communications module and the central location during moments of low-quality connections. Communications through the network can be protected using one or more encryption techniques, such as those techniques provided by the Advanced Encryption Standard (AES), which superseded the Data Encryption Standard (DES), the IEEE 802.1 standard for port-based network security, pre-shared key, Extensible Authentication Protocol (“EAP”), Wired Equivalent Privacy (“WEP”), Temporal Key Integrity Protocol (“TKIP”), Wi-Fi Protected Access (“WPA”), and the like.

While certain examples, aspects, and/or embodiments have been disclosed, it should be appreciated that these are to be non-limiting. In addition, it should be noted that any of the aspects of any of the embodiments could be combined with other aspects in ways not explicitly disclosed herein to create even additional embodiments that would be obvious to those skilled in the art from a reading of the present disclosure. The same goes for variations, alternatives, and other changes.

1. A method of identifying two or more enzymes suitable for fusion into a multifunctional enzyme, comprising:
selecting a first enzyme having a first reactive center;
comparing a second enzyme having a second reactive center to the first enzyme; and
determining that the first and second enzymes are suitable for fusion if the second reactive center is structurally similar to the first reactive center.

2. The method of claim 1, wherein the comparing step comprises determining the reactive center similarity (CRCSim) score of the first and second reactive centers.

3. The method of claim 1, wherein the first and second reactive centers are deemed structurally similar when the CRCSim score is greater than about 0.8.

4. The method of claim 1, wherein the first and second enzymes have different functions.

5. The method of claim 1, wherein the first enzyme and/or second enzyme is a hydrolase.

6. The method of claim 1, wherein the first and second enzymes are stored in and/or selected from a database.

7. A method of designing multifunctional enzymes, comprising:
selecting a first enzyme having a first reactive center having a first function;
identifying a second enzyme having a second reactive center having a second function that is different than the first function of the first reactive center, wherein the second reactive center includes a similar structure of the first reactive center; and
mutating the first enzyme to add the second function to the first reactive center to create a multifunctional enzyme that performs both the first and second functions.

8. The method of claim 7, wherein the similar structure of the first reactive center and the second reactive center comprises similar geometry.

9. The method of claim 7, wherein the first reactive center comprises a first catalytic motif and the second reactive center comprises a second catalytic motif.

10. The method of claim 9, wherein the multifunctional enzyme has a multifunctional reactive center.

11. The method of claim 10, wherein the multifunctional reactive center comprises the first and second catalytic motifs.

12. The method of claim 7, wherein the mutating step comprises mutating a residue within the first reactive center.

13. The method of claim 12, wherein the mutated residue is not a catalytic residue.

14. The method of claim 7, wherein the mutating step comprises designing and/or stabilizing the multifunctional enzyme using a protein hallucinator.

15. The method of claim 14, wherein the protein hallucinator comprises ProteinMPNN.

16. The method of claim 7, wherein the first and second enzymes are stored in and/or selected from a database.

17. The method of claim 7, wherein the amount of similarity between the first and second reactive centers comprises a CRCSim greater than about 0.8.

18. The method of claim 7, wherein the first enzyme and/or second enzyme is a hydrolase.

19. A system for identifying two or more enzymes suitable for fusion into a multifunctional enzyme, comprising:
a computer readable medium including instructions to perform a method comprising:
selecting a first enzyme having a first reactive center;
comparing a second enzyme having a second reactive center to the first enzyme; and
determining that the first and second enzymes are suitable for fusion if the second reactive center is structurally similar to the first reactive center.

20. The system of claim 19, wherein the comparing step comprises determining the reactive center similarity (CRCSim) score of the first and second reactive centers, and wherein the first and second reactive centers are deemed structurally similar when the CRCSim score is greater than about 0.8.
